(19) United States
(12) Patent Application Publication
(10) Pub. No.: US 2015/0152027 A1
(43) Pub. Date: Jun. 4, 2015

(54) INTEGRATED SOLVENT-DEASPHALTING AND FLUID CATALYTIC CRACKING PROCESS FOR LIGHT OLEFIN PRODUCTION

(71) Applicant: Saudi Arabian Oil Company, Dhahran (SA)

(72) Inventors: Raheel Shafi, Manama (BH); Abdennour Bourane, Ras Tanura (SA); Musaed Salem Al-Ghrami, Dhahran (SA); Raed Abudawoud, Dhahran (SA)

(21) Appl. No.: 14/557,687

(22) Filed: Dec. 2, 2014

Related U.S. Application Data
(60) Provisional application No. 61/910,676, filed on Dec. 2, 2013.

(51) Int. Cl. C07C 4/06; C07C 7/10

(52) U.S. Cl. CPC C07C 4/06; C07C 7/10; C07C 2529/80; C07C 2529/08; C07C 2529/40

(57) ABSTRACT

The process described herein broadly comprehends an integrated process that permits the use of straight run residual fractions as a feedstock to produce lighter olefins including propylene. A blend of natural gas condensate and/or naphtha, and heavy oil residue(s), is catalytically cracked to produce a light olefin-rich product stream. In particular, the light natural gas condensate and/or naphtha stream is used as both solvent in a solvent-deasphalting unit and a portion for the feedstock to a fluidized catalytic cracking process. In addition, blending a heavy boiling hydrocarbon stream with a light natural gas condensate or naphtha stream enables efficient cracking in fluidized catalytic cracking process processes while sustaining the heat balance within the cracking unit.

### RELATED APPLICATIONS

The present application claims the benefit of U.S. Provisional Patent Application No. 61/910,676 filed on 2 Dec. 2013, the disclosure of which is incorporated herein by reference in its entirety.

### BACKGROUND OF THE INVENTION

1. Field of the Invention

The present invention relates to an integrated solvent deasphalting and high-severity catalytic cracking process for the production of light olefins, particularly propylene.

2. Description of Related Art

Light olefins are basic intermediates for a large number of processes in the petrochemical industry. They are typically obtained through the thermal cracking (or steam pyrolysis) of petroleum gases and distillates such as naphtha, kerosene or in some instances gas oil. However, thermal cracking typically yields higher rates of ethylene than it does propylene, which has significant market demand.

In particular, there are often shortages of propylene which result in uncertainties in the supply of feedstocks, escalating raw material costs and similar commercial detriments. Additionally, due to uncertainties in the valuations of different hydrocarbon fractions, alternative feedstocks for propylene production are desirable.

Fluidized catalytic cracking is a well-known process that can be tailored to provide relatively higher selectivity of propylene over ethylene, particularly as compared to steam pyrolysis. Common feeds for fluidized catalytic cracking processes include vacuum gas oil, hydrotreated bottoms, hydrocracked bottoms and crude oil residuum. However, these feeds do not typically result in high olefin product yields and require costly pretreatment steps to increase the hydrogen content.

Therefore, a need remains for improved processes and systems for high olefin product yields while minimizing the need to use valuable hydrocarbon fractions from within a refinery.

### SUMMARY OF THE INVENTION

It is an objective of the present invention to provide a process in which alternative feedstocks are cracked using fluidized catalytic cracking to increase the proportion of lighter olefms, in particular propylene.

The process described herein broadly comprehends an integrated process that permits the use of straight run residual fractions as a feedstock to produce lighter olefins including propylene.

For the process described herein a new type of feedstream is provided to the integrated system and process in order to maximize light olefins production. A blend of natural gas condensate and/or naphtha, and heavy oil residue(s), is catalytically cracked to produce a light olefin-rich product stream. In particular, the light natural gas condensate and/or naphtha stream is used as both solvent in a solvent-deasphalting unit and a portion for the feedstock to a fluidized catalytic cracking process. In addition, blending a heavy boiling hydrocarbon stream with a light natural gas condensate or naphtha stream enables efficient cracking in fluidized catalytic cracking process processes while sustaining the heat balance within the cracking unit.

Before being catalytically cracked to a stream rich in light olefins, the light crude oil fractions, such as naphtha and/or natural gas condensates, are used as the solvent in a solvent deasphalting unit, where the metallic and asphaltenic compounds, that could poison the fluidized catalytic cracking catalysts, are removed from the heavy residual oil fraction (such as atmospheric or vacuum distillation residue). This allows obtaining a mixture of the light hydrocarbon fraction and deasphalted heavy feed that has reduced level amounts of metals, sulfur, nitrogen, ash, and Conradson carbon.

The light streams that are blended in the integrated generally comprise hydrocarbons with carbon numbers in the range of 5-7, where the deasphalted oil is solubilized in the solvent. The insoluble pitch will precipitate out of the mixed solution and is separated from the mixture of solvent and deasphalted/demetalized oil in one or more extractors.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

The present process will be described in further detail below and with reference to the attached drawings in which the same or similar reference number is used to refer to the same and similar elements, and in which:

FIG. 1 is a schematic flow diagram of an embodiment of apparatus for fluidized catalytic cracking of a natural gas condensate and/or naptha blended with a heavy oil residue(s) feedstream suitable for production of light olefins including propylene;

FIG. 2 is a simplified schematic illustration of a solvent deasphalting/demetalizing system;

FIG. 3 is a simplified schematic illustration of a downflow fluidized catalytic cracking reactor system; and

FIG. 4 is a simplified schematic illustration of a riser fluidized catalytic cracking reactor system.

### DETAILED DESCRIPTION OF THE INVENTION

Due to increasing demand for propylene, new feedstock sources are desired for its production. The processes and systems described herein utilize feedstock sources for fluidized catalytic cracking including natural gas condensates and petroleum naphthas, along with residual fractions, directly from atmospheric or vacuum distillation bottoms, in an integrated process including solvent deasphalting and fluidized catalytic cracking. The integrated process maximizes the yield of light olefins, including propylene.

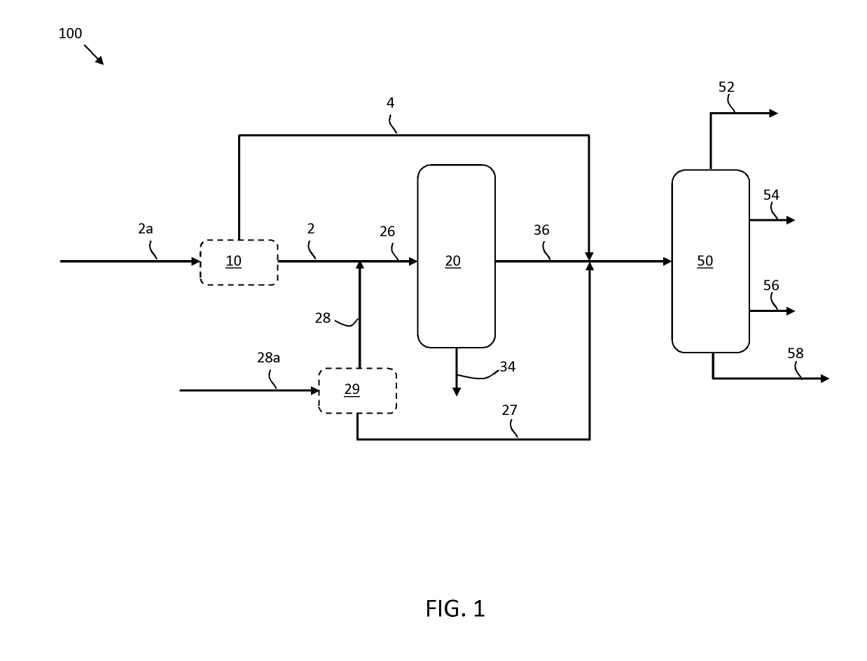

FIG. 1 is a schematic flow diagram including integrated solvent deasphalting and fluidized catalytic cracking processes. The system generally includes a solvent deasphalting/demetalizing zone 20 and a fluidized catalytic cracking reaction and separation zone 50.

In certain embodiments, an initial feedstock 2 is charged to the solvent deasphalting zone 20 along with a solvent feedstock 28 in which it is subjected to conventional solvent deasphalting processes, and an asphalt phase is discharged via outlet 34. The deasphalted/demetalized oil, in combination with the solvent feedstock that is used for deasphalting operations in place of conventional solvent that is typically recycled from the system, is passed via a stream 36 to the fluidized catalytic cracking reaction and separation zone 50. Fluidized catalytic cracking reactions occur under conditions that maximize formation of light olefins, including propylene, and products including light gases 52, olefins 54, gasoline 56 and cycle oil 58 are recovered from a separation zone.

In certain embodiments, an optional splitter 10 is provided which receives all or a portion of an initial feedstock 2a and is in fluid communication with the fluidized catalytic cracking reaction and separation zone 50 to pass a light fraction 4 thereto and the solvent deasphalting zone 20 to pass a heavy fraction 2 thereto. In these embodiments the light fraction 4 is recombined with stream 36 containing deasphalted/demetalized oil and solvent feedstock, and is charged to the fluidized catalytic cracking reaction and separation zone 50.

In certain embodiments, an optional splitter 29 is provided which receives all or a portion of the initial solvent feedstock 28a and is in fluid communication with the fluidized catalytic cracking reaction and separation zone 50 to pass a heavy fraction 27 of the solvent feedstock thereto and the solvent deasphalting zone 20 to pass a light fraction 28 thereto. In these embodiments the light fraction 28 serves as the solvent in the solvent deasphalting/demetalizing zone 20 and the heavy fraction 27 is combined with stream 36 containing deasphalted/demetalized oil and solvent feedstock, and is charged to the fluidized catalytic cracking reaction and separation zone 50.

In additional embodiments (not shown), a relatively small portion of stream 36, particularly all or a portion of C5 and lower components, can be recycled to solvent deasphalting/demetalizing zone 20 or flashed off, since these compounds will not be completely cracked under the herein FCC conditions. For instance, these C5 and lower components derived from Khuff condensate can be recycled or flashed.

Unit operations associated with the fluidized catalytic cracking reaction and separation zone 50 and solvent deasphalting zone 20 are described below. In the interest of clarity, the numerous valves, temperature sensors, electronic process controllers and the like that are customarily employed, and that are well known to those of ordinary skill in the art of solvent deasphalting/demetalizing and fluidized catalyst cracking, are not included in the attached schematic illustration. Accessory systems that are utilized in conventional fluidized catalyst cracking systems such as, for example, air supply, catalyst hoppers, torch oil supply, flue gas handling and heat recovery, fresh and spent catalyst hoppers for storage of make-up and used/equilibrium catalyst that can be added to, or removed from the regenerator, are not shown.

According to the present process and system, the solvent in the solvent deasphalting/demetalizing operation is not separated from the deasphalted/demetalized oil, contrary to conventional solvent deasphalting/demetalizing operations.

Solvent deasphalting/demetalizing is generally carried-out in liquid phase and therefore the temperature and pressure are set accordingly at or below the critical temperature and pressure of the solvent. In a typical two-stage solvent deasphalting/demetalizing process, the temperature is lower in the first stage than that of the second stage to separate the bulk of the asphaltenes. The second stage temperature is set to control the deasphalted/demetalized oil quality and quantity. The temperature has a large impact on the quality and quantity of deasphalted/demetalized oil. An extraction temperature increase will result in a decrease in deasphalted/demetalized oil yield, in turn the deasphalted/demetalized oil will be lighter, less viscous, and contain less metals, asphaltenes, sulfur, and nitrogen. A temperature decrease will have the opposite impact. In general, the deasphalted/demetalized yield decreases with higher quality product by raising the extraction system temperature, and the deasphalted/demetalized yield increases with lower quality product by lowering extraction system temperature.

In conventional solvent deasphalting/demetalizing operations, composition of the solvent is an important process variable. The solubility properties of the solvent increases with increasing critical temperature, C3<iC4<nC4<iC5. An increase in critical temperature of the solvent increases the deasphalted/demetalized oil yield. However, it should be noted that solvents possessing higher critical temperatures have lower selectivity resulting in lower deasphalted/demetalized oil quality. According to certain embodiments of the process herein, in which naphtha or natural gas condensates are used as a solvent in the solvent deasphalting/demetalizing operation, all or a portion of the heavy-end components of the naphtha or natural gas condensates are removed by fractionation and the lighter portion of the stream is used in the solvent deasphalting/demetalizing process.

The ratio of the light fraction (solvent feedstock) to the heavy fraction (oil feedstock) entering the deasphalting/demetailzing unit impacts selectivity and to a lesser degree yield of deasphalted/demetalized oil. The major effect of light to heavy stream ratio is that a higher ratio results in a higher quality of the deasphalted/demetalized oil for a given fixed deasphalted/demetalized yield. A high solvent (light fraction) to heavy fraction ratio is preferred in certain embodiments due to of enhanced selectivity. In the process and system herein, ratio of the light to the heavy fraction entering the deasphalting/demetailzing unit is determined as a function of the coke that will be produced during the cracking reaction within the fluidized catalytic cracking reaction, as described further herein. The solvent-to-oil ratio is in the range of about 5:1 to about 50:1, in certain embodiments in the range of about 5:1 to about 25:1, and in further embodiments in the range of about 5:1 to about 15:1. The ratio of the light to heavy fraction of the hydrocarbon mixture fed to the fluidized catalytic cracking process is manipulated not only to ensure generation of sufficient coke in the cracking reactions so as to maintain a heat balance in the fluidized catalytic cracking unit, but also to facilitate optimal conversion to obtain high light olefins yield.

The solvent deasphalting unit is operated at a pressure that is sufficiently high to maintain the solvent in the liquid phase within the extraction system. The pressure is typically considered as a variable and is typically not altered unless the solvent composition varies.

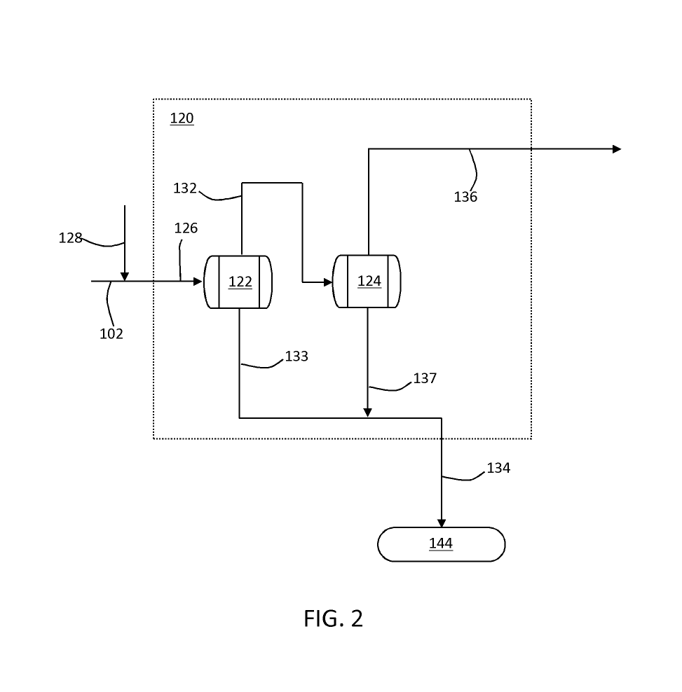

FIG. 2 is a simplified schematic illustration of a solvent deasphalting/demetalizing system. Exemplary unit operations within solvent deasphalting zone 20 of the integrated system described with respect to FIG. 1 are shown. While the system and process is shown with primary and secondary extraction zones, it should be appreciated that one extraction zone can be used, or alternatively more than two extraction zones can be used.

A solvent deasphalting zone 120 typically includes a primary extraction zone 122 and a secondary extraction zone 124. Primary extraction zone 122 includes an inlet for receiving a combined stream 126 including the hydrocarbon feedstock 102 (or a heavy portion thereof in embodiments in which the optional splitter 110 is used) and a solvent feedstock 128. Primary extraction zone 122 also includes an outlet for discharging a primary deasphalted/demetalized oil phase 132 and one or more outlets for discharging a primary asphalt phase 133. Secondary extraction zone 122 typically includes an inlet for receiving the primary deasphalted/demetalized oil phase 132, an outlet for discharging a secondary deasphalted/demetalized oil phase 136 and an outlet for discharging a secondary asphalt phase 137. The combined primary asphalt phase 133 and secondary asphalt phase 137, stream 134, is passed to an asphalt pool 144.

According to the process herein, the solvent feedstock 128 is selected so as to be suitable not only as a solvent for facilitating precipitation of the asphalt phase 134 of the feedstock 102, but also to serve as a reactant in the downstream fluidized catalytic cracking process to yield fluidized catalytic cracking products olefins, gasoline and cycle oil, with a maximized propylene yield. Therefore the solvent deasphalting zone 120 does not require separate solvent recovery/recycle steps or unit operations since the solvent component is part of the combined stream 136 that is the feed to the fluidized catalytic cracking reaction.

In general, during fluidized catalytic cracking processes, petroleum derived hydrocarbons are catalytically cracked with an acidic catalyst maintained in a fluidized state, which is regenerated on a continuous basis. The main product from such processes has conventionally been gasoline. Other products are also produced in smaller quantities via fluidized catalytic cracking such as liquid petroleum gas and cycle oil. Coke deposited on the catalyst is burned off at high temperatures and in the presence of air prior to recycling regenerated catalyst back to the reaction zone. As discussed above, a shift in market demands has led to use of fluidized catalytic cracking for production of light olefins, including propylene, as a primary product. These operations have significant economic advantages, particularly with respect to oil refineries that are highly integrated with petrochemical production facilities.

According to the processes and systems herein, a high-severity fluid catalytic cracking process is used to facilitate conversion of the feed into a product rich in light olefins. For instance, a PetroFCC process (disclosed by UOP, a Honeywell International Inc. company) can be suitable for the present processes and systems. In general, suitable high-severity fluid catalytic cracking processes result in minimization of olefin consuming reactions thereby maximizing the yield of light olefin products such as propylene.

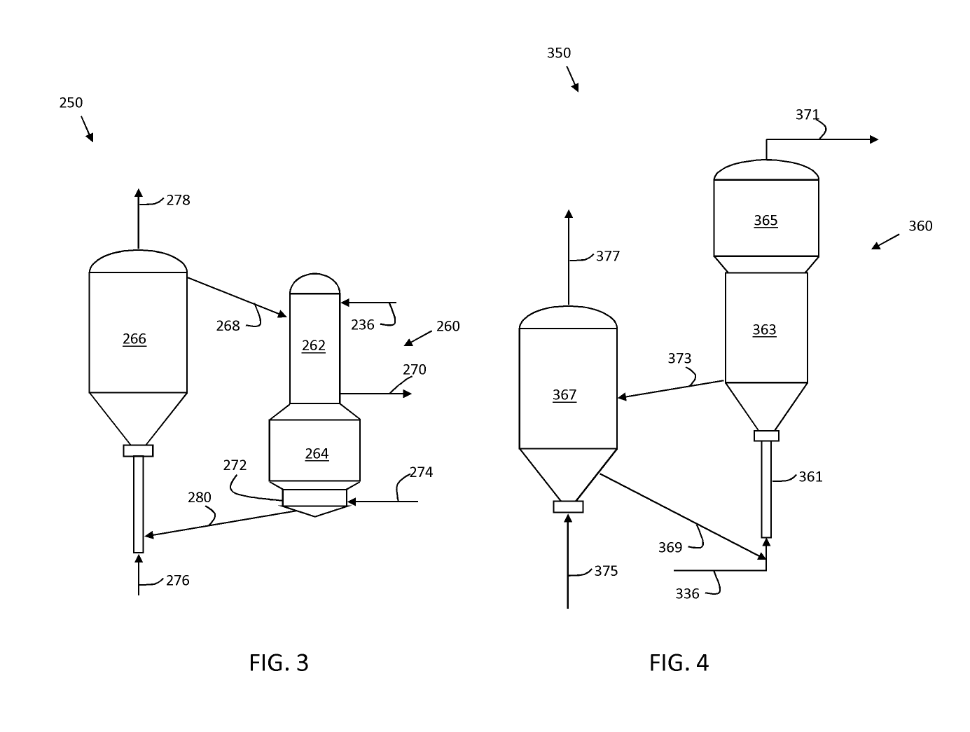

In certain embodiments, a fluidized catalytic cracking unit configured with a downflow reactor is provided that operates under conditions that promote formation of olefins, particularly propylene, and that minimize olefin-consuming reactions including hydrogen-transfer reactions. FIG. 3 is a simplified schematic illustration of a downflow fluidized catalytic cracking unit. Exemplary unit operations within fluidized catalytic cracking zone 50 of the integrated system described with respect to FIG. 1 are shown. A fluidized catalytic cracking unit 250 includes a reactor/separator 260 having a reaction zone 262 and a separation zone 264. Fluidized catalytic cracking unit 250 also includes a regeneration zone 266 for regenerating spent catalyst. In particular, a charge 236 (which is the combined stream of deasphalted/demetalized and solvent feed, and in certain embodiments recombined with light ends of the feed and/or heavy ends of the solvent as described above) is introduced to the reaction zone, in certain embodiments accompanied by steam or other suitable gas for atomization of the feed (not shown). An effective quantity of heated fresh or hot regenerated solid cracking catalyst particles from regeneration zone 266 is conveyed to the top of reaction zone 262 also transferred, e.g., through a downwardly directed conduit or pipe 268, commonly referred to as a transfer line or standpipe, to a withdrawal well or hopper (not shown) at the top of reaction zone 262. Hot catalyst flow is typically allowed to stabilize in order to be uniformly directed into the mix zone or feed injection portion of reaction zone 262. The charge 236 is injected into a mixing zone through feed injection nozzles typically situated proximate to the point of introduction of the regenerated catalyst into reaction zone 262. These multiple injection nozzles result in the catalyst and oil mixing thoroughly and uniformly. Once the charge contacts the hot catalyst, cracking reactions occur.

The reaction vapor of hydrocarbon cracked products, unreacted feed and catalyst mixture quickly flows through the remainder of reaction zone 262 and into the rapid separation zone 264 at the bottom portion of reactor/separator 260. Cracked and uncracked hydrocarbons are directed through a conduit or pipe 270 to a conventional product recovery section known in the art to yield fluidized catalytic cracking products olefins, gasoline and cycle oil, with a maximized propylene yield. If necessary for temperature control, a quench injection can be provided near the bottom of reaction zone 262 immediately before the separation zone 264. This quench injection quickly reduces or stops the cracking reactions and can be utilized for controlling cracking severity.

The reaction temperature, i.e., the outlet temperature of the downflow reactor, can be controlled by opening and closing a catalyst slide valve (not shown) that controls the flow of regenerated catalyst from regeneration zone 266 into the top of reaction zone 262. The heat required for the endothermic cracking reaction is supplied by the regenerated catalyst. By changing the flow rate of the hot regenerated catalyst, the operating severity or cracking conditions can be controlled to produce the desired yields of light olefinic hydrocarbons. A stripper 272 is also provided for separating oil from the catalyst, which is transferred to regeneration zone 266. The catalyst from separation zone 264 flows to the lower section of the stripper 272 that includes a catalyst stripping section into which a suitable stripping gas, such as steam, is introduced through streamline 274. The stripping section is typically provided with several baffles or structured packing (not shown) over which the downwardly flowing catalyst 280 passes counter-currently to the flowing stripping gas. The upwardly flowing stripping gas, which is typically steam, is used to “strip” or remove any additional hydrocarbons that remain in the catalyst pores or between catalyst particles. The stripped or spent catalyst is transported by lift forces from the combustion air stream 276 through a lift riser of the regeneration zone 264. This spent catalyst, which can also be contacted with additional combustion air, undergoes controlled combustion of any accumulated coke. Flue gases are removed from the regenerator via conduit 278. In the regenerator, the heat produced from the combustion of the by-product coke is transferred to the catalyst raising the temperature required to provide heat for the endothermic cracking reaction in the reaction zone 262. According to the process herein, since the light solvent feedstock is combined with the heavy feedstock as the feed 236, the solvent to oil ratio in the initial solvent deasphalting/demetalizing process is selected so as to provide sufficient coking of the catalyst to provide the heat balance during regeneration.

In one embodiment, a suitable fluidized catalytic cracking unit 250 that can be employed in the integrated solvent deasphalting and high-severity catalytic cracking system and process described herein can be similar to those described in US Patent Number 6,656,346, and US Patent Publication Number 2002/0195373, both of which are incorporated herein by reference. Important properties of downflow reactors include introduction of feed at the top of the reactor with downward flow, shorter residence time as compared to riser reactors, and high catalyst to oil ratio, e.g., in the range of about 20:1 to about 30:1.

In general, the operating conditions for the reactor of a suitable downflow FCC unit include:

reaction temperature of about 500° C. to about 650° C., in certain embodiments about 550° C. to about 630° C., and in further embodiments about 570° C. to about 610° C.;

reaction pressure of about 1 Kg/cm² to about 5 Kg/cm², in certain embodiments of about 1 Kg/cm² to about 3 Kg/cm², in further embodiments of about 1 Kg/cm² to about 2 Kg/cm²;

contact time (in the reactor) of about 0.1 seconds to about 10 seconds, in certain embodiments of about 0.1 seconds to about 5 seconds, in further embodiments of about 0.2 seconds to about 0.8 seconds; and

a catalyst to feed ratio of about 2:1 to about 40:1, in certain embodiments of about 2:1 to about 30:1, in further embodiments of about 2:1 to about 20:1.

In certain embodiments, a fluidized catalytic cracking unit configured with a riser reactor is provided that operates under conditions that promote formation of olefins, particularly propylene, and that minimize olefin-consuming reactions including hydrogen-transfer reactions. FIG. 4 is a simplified schematic illustration of a riser fluidized catalytic cracking unit. Exemplary unit operations within fluidized catalytic cracking zone 50 of the integrated system described with respect to FIG. 1 are shown. A fluidized catalytic cracking unit 350 which includes a riser reactor and can be used in the herein integrated solvent deasphalting and high-severity catalytic cracking system and process. Fluidized catalytic cracking unit 350 includes a reactor/separator 360 having a riser portion 361, a reaction zone 363 and a separation zone 365. Fluidized catalytic cracking unit 350 also includes a regeneration vessel 367 for regenerating spent catalyst. A charge 336 (which is the combined stream of deasphalted/demetalized and solvent feed, and in certain embodiments recombined with light ends of the feed and/or heavy ends of the solvent as described above) is introduced to the reaction zone, in certain embodiments accompanied by steam or other suitable gas for atomization of the feed (not shown). The charge 336 is admixed and intimately contacted with an effective quantity of heated fresh or regenerated solid cracking catalyst particles which are conveyed via a conduit 369 from regeneration vessel 367. The feed mixture and the cracking catalyst are contacted under conditions to form a suspension that is introduced into the riser 361. In a continuous process, the mixture of cracking catalyst and hydrocarbon feedstock proceed upward through the riser 361 into reaction zone 363. In riser 361 and reaction zone 363, the hot cracking catalyst particles catalytically crack relatively large hydrocarbon molecules by carbon-carbon bond cleavage.

During the reaction, as is conventional in fluidized catalytic cracking operations, the cracking catalysts become coked and hence access to the active catalytic sites is limited or nonexistent. Reaction products are separated from the coked catalyst using any suitable configuration known in a fluidized catalytic cracking units, generally referred to as the separation zone 365 in a fluidized catalytic cracking unit 350, for instance, located at the top of the reactor 360 above the reaction zone 363. The separation zone can include any suitable apparatus known to those of ordinary skill in the art such as, for example, cyclones. The reaction product is withdrawn through conduit 371. Catalyst particles containing coke deposits from fluid cracking of the hydrocarbon feedstock pass through a conduit 373 to regeneration zone 367. According to the process herein, since the light solvent feedstock is combined with the heavy feedstock as the feed 336, the solvent to oil ratio in the initial solvent deasphalting/demetalizing process is selected so as to provide sufficient coking of the catalyst to provide the heat balance during regeneration.

In regeneration zone 367, the coked catalyst comes into contact with a stream of oxygen-containing gas, e.g., pure oxygen or air, which enters regeneration zone 367 via a conduit 375. The regeneration zone 367 is operated in a configuration and under conditions that are known in typical a fluidized catalytic cracking operations. For instance, regeneration zone 367 can operate as a fluidized bed to produce regeneration off-gas comprising combustion products which is discharged through a conduit 377. The hot regenerated catalyst is transferred from regeneration zone 367 through conduit 369 to the bottom portion of the riser 361 for admixture with the hydrocarbon feedstock and noted above.

In one embodiment, a suitable a fluidized catalytic cracking unit 350 that can be employed in the integrated solvent deasphalting and high-severity catalytic cracking system and process described herein can be similar to that described in U.S. Pat. Nos. 7,312,370, 6,538,169, and 5,326,465, which are incorporated herein by reference.

In general, the operating conditions for the reactor of a suitable riser fluidized catalytic cracking unit include:

reaction temperature of about 500° C. to about 650° C., in certain embodiments about 550° C. to about 630° C., and in further embodiments about 570° C. to about 610° C.;

reaction pressure of about 1 Kg/cm² to about 5 Kg/cm², in certain embodiments of about 1 Kg/cm² to about 3 Kg/cm², in further embodiments of about 1 Kg/cm² to about 2 Kg/cm²;

contact time (in the reactor) of about 0.7 seconds to about 10 seconds, in certain embodiments of about 1 second to about 5 seconds, in further embodiments of about 1 second to about 2 seconds ; and

a catalyst to feed ratio of about 2:1 to about 40:1, in certain embodiments of about 2:1 to about 25:1, in further embodiments of about 2:1 to about 15:1.

The catalyst or catalyst systems that can be used in the process described herein are not limited. A catalyst that is suitable for the particular charge and the desired product is conveyed to the FCC reactor within the FCC reaction and separation zone. In certain embodiments, to promote formation of olefins and minimize olefin-consuming reactions, such as hydrogen-transfer reactions, an FCC catalyst mixture is used in the FCC reaction and separation zone, including an FCC base catalyst and an FCC catalyst additive.

In particular, a matrix of a base cracking catalyst can include one or more clays such as kaolin, montmorilonite, halloysite and bentonite, and/or one or more inorganic porous oxides such as alumina, silica, boria, chromia, magnesia, zirconia, titania and silica-alumina. The base cracking catalyst preferably has a bulk density of 0.5 g/ml to 1.0 g/ml, an average particle diameter of 50 microns to 90 microns, a surface area of 50 m²/g to 350 m²/g and a pore volume of 0.05 ml/g to 0.5 ml/g.

A suitable catalyst mixture contains, in addition to a base cracking catalyst, an additive containing a shape-selective zeolite. The shape selective zeolite referred to herein means a zeolite whose pore diameter is smaller than that of Y-type zeolite, so that hydrocarbons with only limited shape can enter the zeolite through its pores. Suitable shape-selective zeolite components include ZSM-5 zeolite, zeolite omega, SAPO-5 zeolite, SAPO-11 zeolite, SAPO34 zeolite, and pentasil-type aluminosilicates. The content of the shape-selective zeolite in the additive is generally in the range of 20 to 70 wt %, and preferably in the range of 30 to 60 wt %.

The additive preferably has a bulk density of 0.5 g/ml to 1.0 g/ml, an average particle diameter of 50 microns to 90 microns, a surface area of 10 m²/g to 200 m²/g and a pore volume of 0.01 ml/g to 0.3 ml/g.

A percentage of the base cracking catalyst in the catalyst mixture can be in the range of 60 to 95 wt % and a percentage of the additive in the catalyst mixture is in a range of 5 to 40 wt %. If the percentage of the base cracking catalyst is lower than 60 wt % or the percentage of additive is higher than 40 wt %, high light-fraction olefin yield cannot be obtained, because of low conversions of the feed oil. If the percentage of the base cracking catalyst is higher than 95 wt %, or the percentage of the additive is lower than 5 wt %, high light-fraction olefin yield cannot be obtained, while high conversion of the feed oil can be achieved.

The initial heavy feedstock for use in above-described systems and processes can be a straight run residue obtained from atmospheric distillation and/or vacuum distillation. In general, the feedstock contains hydrocarbons having boiling point greater than about 380° C. in the case of atmospheric residue, and greater than about 540° C. in the case of vacuum residue. The source of the residues can be crude oil, synthetic crude oil, bitumen, oil sand, shell oil, coal liquids, or a combination including one of the foregoing sources.

In a process using the system of FIG. 1, all or a portion of the light stream 28a (naphtha or natural gas condensate of carbon range starting at C5 or above) is first fed to an optional splitter 29 to remove a heavy portion 27 thereof, if required to optimize the solvent deasphalting/demetalizing operation. The light portion 28 is admixed with a heavy oil fraction 2, such as atmospheric or vacuum residue. The resulting mixture 26 is then transferred to the solvent deasphalting/demetalizing zone 20, for instance, which can be operated in a manner similar to what is reported in B. M. Yezhov, A. S. Aigenson, G. A. Berg, F. Kh. Malikov, Yu. S. Sabadash and G. A. Vorms, Hydrocarbon Separation, including Engineering Developments, 1971, 8^{th} World Petroleum Congress, 221-227, which is incorporated herein by reference, in which a gasoline-type stream is used as the solvent to extract asphaltenes from a heavy hydrocarbon fraction. The asphaltenes 34 are rejected from the hydrocarbon mixture and the resultant mixture 36 is directly sent to the fluidized catalytic cracking zone 50. In embodiments in which the optional splitter is used to remove a heavy portion of the solvent feedstream prior to mixing with the heavy fraction 2, the heavy portion 27 is recombined with the feed passed to the fluidized catalytic cracking zone 50.

Advantageously, the processes and systems described herein accommodate the high demand for light olefins with the use of alternative feedstocks including hydrocarbons in lighter boiling-ranges than those which are usually used as fluidized catalytic cracking feeds. These lighter feeds include natural gas condensates and petroleum naphthas.

The problems that arise when lighter feedstocks are used in the fluidized catalytic cracking feeds process is the low coking on the catalyst. This lower amount of coke is not sufficient to provide the heat for catalytic regeneration in the fluidized catalytic cracking process. Therefore, an additional heat source would typically be required for the fluidized catalytic cracking process to operate properly. However, according to the process herein, blending an effective quantity of a very heavy fraction, such as atmospheric or vacuum residue, with the light fraction(s) can result in sufficient coke for catalyst regeneration.

Using the lighter feeds as blends, however, does not counter the negative impacts that result from use of straight run residues (i.e., those directly from atmospheric an./or vacuum distillation) such as catalyst poisoning due to metal and asphaltenic content of the heavy oil fractions. In order to avoid poisoning the fluidized catalytic cracking catalyst these compounds must be removed from the heavy fraction, which leads to a better overall process performance and longer catalyst life. According to the process herein, these metals and asphaltenes are removed by a tightly integrated solvent deasphalting/demetalizing process, in which the solvent also serves as the feedstock blend to increase propylene yield.

It is to be understood that the present processes contemplate use of the naphtha or natural gas condensate as a solvent and reactant. This is in contrast to known processes such as that disclosed in Bartilucci et al. U.S. Pat. No. 5,000,838 in which naphtha that may remain in a deasphalted oil does not serving as a reactant to produce light olefins. In that process, the boiling range of the cracking product is 100-400° F. (37.8-204.4° C.), whereas light olefins (ethylene and propylene) boil in the range of −155 to −54° F. (−103.9 to −47.8° C.) indicating that the fluidized catalytic cracking process described therein is operated at conditions through which light olefins cannot be produced and that any naphtha in the cracking feed does not react at all, since one would expect the boiling range of the product to be less than the feed if cracking reactions were to occur.

### EXAMPLE

An atmospheric residue derived from atmospheric distillation of a crude oil was mixed with gas condensate at a volumetric condensate:residue ratio of 10:1 for partial deasphalting. The mixture was stirred at room temperature, them maintained for a few hours to settle, and filtered to separate the precipitated material. About 5wt % of the atmospheric residue was precipitated. The properties of the atmospheric residue, condensate and obtained deasphalted mixture are given in Table 1 below.

TABLE 1

| | Atmospheric | Gas | Deasphalted |
|---|---|---|---|
| | Residue | Condensate | Mixture |
| Gravity, Specific 60/60 ° F. | 0.9651 | 0.7721 | 0.7796 |
| Sulfur, Total Wt % | 3.2 | 0.0521 | 0.0560 |
| Vanadium, ppm | 29 | — | 1.4 |
| Microcarbon Residue, wt % | 6.2 | 0.01 | 0.31 |
| Nickel, ppm | 9 | — | <2 |

Catalytic cracking of the deasphalted mixture was conducted using a micro activity testing (MAT) unit well known to a person of ordinary skill in the art at 600° C. and a C/O ratio of ˜4. The catalyst contains a mixture of Y zeolite and ZSM-5. The cracking of the gas condensate alone was also similarly performed and the products yields difference (relative) are shown in Table 2. As seen, more coke can be produced from the mixture feed while maintaining propylene yield.

TABLE 2

| Product | Deasphalted Mixture Vs Gas |
|---|---|
| Yields | condensate relative difference (%) |
| Dry gas | +3 |
| LPG | −6 |
| propylene | −6 |
| gasoline | −1 |
| LCO | +20 |
| HCO | +17 |
| Coke | +28 |

The process and system of the present invention have been described above and in the attached drawings; however, modifications will be apparent to those of ordinary skill in the art and the scope of protection for the invention is to be defined by the claims that follow.

1. An integrated process for production of propylene comprising:
mixing a residual oil feedstream with a solvent in quantities and under conditions sufficient to precipitate at least a fraction of the asphaltenes, thereby producing a mixture of solvent and deasphalted residual oil, and precipitated asphaltenes, and;
subjecting the mixture of solvent and deasphalted residual oil to fluidized catalytic cracking reactions, wherein both the solvent and the deasphalted residual oil from the mixture of solvent and deasphalted residual oil serve as reactants in the fluidized catalytic cracking reaction.

2. The process as in claim 1 wherein the mixture of solvent and deasphalted residual oil is not subjected to solvent recovery prior to fluidized catalytic cracking reactions.

3. The process as in claim 1 wherein subjecting the mixture of solvent and deasphalted residual oil to fluidized catalytic cracking reactions occurs under high-severity fluid catalytic cracking conditions to facilitate conversion of reactants into a product rich in light olefins

4. The process as in claim 1, wherein subjecting the mixture of solvent and deasphalted residual oil to fluidized catalytic cracking reactions occurs in a downflow fluid catalytic cracking unit.

5. The process as in claim 4, wherein the reaction temperature in the fluid catalytic cracking unit is about 500° C. to about 650° C.

6. The process as in claim 4, wherein the reaction pressure in the fluid catalytic cracking unit is about 1 Kg/cm2 to about 5 Kg/cm2.

7. The process as in claim 4 wherein contact time between reactants and catalyst in the fluid catalytic cracking unit is about 0.1 seconds to about 10 seconds.

8. The process as in claim 4 wherein a catalyst to feed ratio in the fluid catalytic cracking unit is about 2:1 to about 40:1.

9. The process as in claim 1, in which a portion of the C5 and lower components of the mixture of solvent and deasphalted residual oil are removed prior to fluidized catalytic cracking reactions.

10. The process as in claim 9, wherein a portion of the removed C5 and lower components are recycled as a at least a portion of solvent for the step of mixing the residual oil feedstream with the solvent.

11. The process as in claim 1, wherein the solvent in the step of mixing the residual oil feedstream is naphtha or natural gas condensate of carbon range starting at C5 or above.

12. The process as in claim 1, wherein the solvent in the step of mixing the residual oil feedstream is a light portion of an initial light stream, and a heavy portion of the initial light stream is admixed with the mixture of solvent and deasphalted residual oil for fluidized catalytic cracking reactions.

13. The process as in claim 12, wherein the initial light stream is naphtha or natural gas condensate of carbon range starting at C5 or above.

14. The process as in claim 1, wherein the residual oil in the step of mixing the residual oil feedstream is a heavy portion of an initial heavy stream, and a light portion of the initial heavy stream is admixed with the mixture of solvent and deasphalted residual oil for fluidized catalytic cracking reactions.

15. The process as in claim 1, wherein the residual oil feedstream is a straight run residue obtained from atmospheric distillation and/or vacuum distillation.
